(19) United States
(12) Patent Application Publication
(10) Pub. No.: US 2022/0072270 A1
(43) Pub. Date: Mar. 10, 2022

(54) ACUTE KIDNEY INJURY MONITORING

(71) Applicant: Covidien LP, Mansfield, MA (US)

(72) Inventors: Jacob D. Dove, Lafayette, CO (US); Søren Aasmul, Holte (DK); William S. Smith, Wheat Ridge, CO (US); David J. Miller, Austin, TX (US); Jesper Svenning Kristensen, Holte (DK)

(21) Appl. No.: 17/466,488

(22) Filed: Sep. 3, 2021

Related U.S. Application Data
(60) Provisional application No. 63/074,763, filed on Sep. 4, 2020.

(51) Int. Cl. A61M 25/00; A61M 25/10

(52) U.S. Cl. CPC A61M 25/0017; A61M 25/10; A61M 2025/1047; A61M 2205/35; A61M 2202/0496; A61M 2210/1085; A61M 2205/3327; A61M 2205/3334; A61M 2210/1082

(57) ABSTRACT

An example device includes memory configured to store an observer and processing circuitry communicatively coupled to the memory. The processing circuitry is configured to receive, from an oxygen sensor, an oxygen sensor signal indicative of an amount of dissolved oxygen in a fluid, wherein the oxygen sensor is located in a distal portion of a catheter or distal to a distal end of the catheter, and wherein the fluid flows to the oxygen sensor from a location within a patient. The processing circuitry is configured to determine, based on the oxygen sensor signal, a measurement of the amount of dissolved oxygen in the fluid. The processing circuitry is configured to apply the observer to the measurement of the amount of dissolved oxygen in the fluid. The processing circuitry is configured to determine, based on the observer, an estimate of an amount of dissolved oxygen in the fluid at the location.

This application claims the benefit of U.S. Provisional Application No. 63/074,763, entitled, “ACUTE KIDNEY INJURY MONITORING” and filed Sep. 4, 2020, the entire contents of which are hereby incorporated by reference.

### TECHNICAL FIELD

This disclosure relates to patient monitoring.

### BACKGROUND

Medical devices, such as catheters, may be used to assist a patient in voiding their bladder. In some instances, such catheters may be used during and/or after surgery. In the case of using a catheter to assist a patient in voiding their bladder, a Foley catheter is a type of catheter that may be used for longer time periods than a non-Foley catheter. Some Foley catheters are constructed of silicon rubber and include an anchoring member, which may be an inflatable balloon, that may be inflated in a bladder of a patient so a proximal end of the catheter does not slip out of the bladder.

### SUMMARY

In general, the disclosure describes devices, systems, and techniques for renal monitoring (also referred to herein as kidney function monitoring) of a patient based on an oxygen content of a fluid (e.g., urine) from the patient. The oxygen content may be used to detect one or conditions indicative of acute kidney injury (AKI) of the patient or a risk the patient will develop AKI. Devices, systems, and techniques described herein apply an observer from control theory to model a catheter and the oxygen equilibration through the catheter wall to estimate oxygen content of a fluid in a location within a patient based on a measurement of oxygen content in the fluid external to the patient and removed from the patient via the catheter. The oxygen content of a fluid removed from a bladder of the patient can be indicative of the oxygenation status of the one or more kidneys of the patient, which can indicate whether the patient is at risk of developing AKI.

In one example, this disclosure is directed to a method including receiving, from an oxygen sensor, an oxygen sensor signal indicative of an amount of dissolved oxygen in a fluid, wherein the oxygen sensor is located in a distal portion of a catheter or distal to a distal end of the catheter, and wherein the fluid flows to the oxygen sensor from a location within a patient; determining, by processing circuitry and based on the oxygen sensor signal, a measurement of the amount of dissolved oxygen in the fluid; applying, by processing circuitry, an observer to the measurement of the amount of dissolved oxygen in the fluid; and determining, by the processing circuitry and based on the observer, an estimate of an amount of dissolved oxygen in the fluid at the location within the patient.

In another example, this disclosure is directed to a device including memory configured to store an observer and processing circuitry communicatively coupled to the memory, the processing circuitry being configured to: receive, from an oxygen sensor, an oxygen sensor signal indicative of an amount of dissolved oxygen in a fluid, wherein the oxygen sensor is located in a distal portion of a catheter or distal to a distal end of the catheter, and wherein the fluid flows to the oxygen sensor from a location within a patient; determine, based on the oxygen sensor signal, a measurement of the amount of dissolved oxygen in the fluid; apply the observer to the measurement of the amount of dissolved oxygen in the fluid; and determine, based on the observer, an estimate of an amount of dissolved oxygen in the fluid at the location within the patient.

In another example, a non-transitory computer-readable storage medium having instructions stored thereon, which, when executed, cause processing circuitry to receive, from an oxygen sensor, an oxygen sensor signal indicative of an amount of dissolved oxygen in a fluid, wherein the oxygen sensor is located in a distal portion of a catheter or distal to a distal end of the catheter, and wherein the fluid flows to the oxygen sensor from a location within a patient; determine, based on the oxygen sensor signal, a measurement of the amount of dissolved oxygen in the fluid; apply an observer to the measurement of the amount of dissolved oxygen in the fluid; and determine, based on the observer, an estimate of an amount of dissolved oxygen in the fluid at the location within the patient.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a diagram illustrating an example catheter.

FIG. 2 is a diagram illustrating an example cross-sectional view of the catheter of FIG. 1, the cross-sections being taken along lines 2-2 of FIG. 1.

FIG. 3 is a block diagram of an example external device that may be used with a medical device according to example techniques of this disclosure.

FIG. 4 is a graph illustrating pO₂ measurements of water that was initially set to ˜43 mmHg and allowed to flow through a silicone Foley catheter at different flow rates.

FIG. 5 is a conceptual diagram of an example observer.

FIG. 6 is a conceptual diagram illustrating a portion of an example catheter.

FIG. 7 is a flow diagram illustrating example observer techniques of this disclosure.

### DETAILED DESCRIPTION

Acute kidney injury (AKI) is a complication that may occur after some medical procedures, such as some cardiac surgeries, e.g., coronary artery bypass grafting (CABG). AKI may also occur after other surgeries that are lengthy and involve significant blood loss or fluid shifts. For example, a body of a surgery patient may alter where their blood is directed which may lead to hypoxia of a kidney. A cause of surgery-associated AKI is hypoxia of the kidneys, which may cause an ischemia reperfusion injury in a kidney of the patient. This ischemia reperfusion injury may cause degradation of renal function of the patient. The degradation of renal function may cause an accumulation of waste products in the bloodstream, which may delay the patient's recovery from the surgery and lead to more extended hospital stays and may even lead to further complications.

The present disclosure describes example devices and systems that are configured to monitor kidney function of a patient, such as a patient who is undergoing or who has undergone such surgeries, based on an oxygen content of a fluid (e.g., urine) removed from a bladder of the patient. The monitoring of kidney function may help reduce occurrences of AKI by providing clinicians with an assessment of the risk that a specific patient may develop AKI. This may facilitate a clinician intervening prior to the patient developing AKI. For example, a clinician may initiate or make changes to hemodynamic management (e.g., blood pressure management, fluid management, blood transfusions, etc.), make changes to cardiopulmonary bypass machine settings, or avoid providing nephrotoxic drugs. Post operatively, a clinician may intervene with a Kidney Disease: Improving Global Outcomes (KDIGO) bundle or an AKI care bundle.

While systemic vital signs like cardiac output, blood pressure, and hematocrit may be useful for monitoring the kidney function of a patient (also referred to herein as renal monitoring), it may also be useful to monitor the oxygenation status of the kidneys in order to limit, reduce the severity of, or even prevent AKI. The amount of dissolved oxygen in a urine of a patient may be indicative of kidney function or kidney health. Dissolved oxygen in a patient's urine and bladder may correlate to perfusion and/or oxygenation of the kidneys, which is indicative of kidney performance. Accurate monitoring of the oxygenation status of the kidneys can be challenging due to the inaccessibility of the kidneys. Near-Infrared spectroscopy (NIRS) measures regional oximetry, and has some utility in babies and relatively slender adults in measuring oxygenation of the kidneys, but may not have the depth of penetration and specificity required for some patients. The present disclosure describes processing circuitry configured to apply an observer from control theory to model a catheter and the oxygen equilibration through the catheter wall so as to enable the estimation of oxygen content of a fluid in a location within a patient based on a measurement of oxygen content in the fluid external to the patient and removed from the patient via a catheter.

In some examples, a medical system described herein includes at least one sensor configured to sense a parameter of a fluid of interest, such as urine in the case of kidney function monitoring. In some examples, the at least one sensor configured to sense a parameter of a fluid of interest may not be a part of a medical device (e.g., a catheter), but may be distal to a distal end of the medical device or inserted into a lumen of the medical device. In some examples, the at least one sensor includes an oxygen sensor configured to sense an oxygen content in a fluid sample and generate an oxygen sensor signal indicative of the oxygen content in the fluid sample. In some examples, the at least one sensor further includes a flow sensor configured to sense a rate of flow of the fluid and to generate a flow sensor signal indicative of the rate of flow of the fluid.

While urine, bladders, and AKI are primarily referred to herein to describe the example medical devices, in other examples, the medical device may be used with other target locations in a patient, such as intravascular locations, and to monitor fluids of interest other than urine and/or other patient conditions other than kidney function. In addition, while catheters are primarily referred to herein, in other examples, the medical device can have another configuration. As discussed in further detail below, in some examples, an example medical device includes a dissolved gas sensor, such as a dissolved oxygen sensor configured to sense an amount of oxygen dissolved in the urine (e.g., oxygen partial pressure (pO₂)) in urine being output from the medical device and/or a flow sensor configured to sense a rate of flow of the urine through the medical device, from which a device may be able to determine oxygenation status of the one or both kidneys of the patient.

Example parameters of interest sensed by a sensor described herein include, but are not limited to, any one or more of an amount of dissolved oxygen, urine flow rate, urine concentration, urine electrical conductivity, urine specific gravity, urine biomarkers, amount of dissolved carbon dioxide in the urine, urine pH, bladder or abdominal pressure, bladder temperature, urine color, urine turbidity, urine creatinine, urine electrical conductivity, urine sodium, or motion from an accelerometer or other motion sensor. In some cases, it may be desirable to sense one or more of these parameters relatively close to the kidneys as possible because when sensors are positioned further away from the kidneys, the risk of introducing noise or losing signal strength increases and/or the risk of the concentration or integrity of a substance of interest in the fluid of interest (e.g., urine) changing prior to being sensed by the sensor may increase. However, an electrical, optical or radio frequency signal representative of a parameter sensed close to the kidneys, may be affected by noise and/or loss of signal strength as the signal travels from a sensor close to the kidneys to a device that may process the signal and display information regarding the sensed parameter. As another example, in the case of a Foley catheter, it may be desirable to sense one or more of these parameters at the proximal end of the Foley catheter (e.g., in the bladder of the patient). However, placing these sensors at the proximal end of the catheter may increase the size and stiffness of the catheter and, as a result, may undermine the patient comfort or deliverability of the catheter. By design, a Foley catheter is configured to be small and flexible, such that it can be inserted through the urethra and into the bladder of a patient. If a Foley catheter were stiffer, then it may be more difficult to comfortably insert the catheter into the bladder of the patient. In some examples, an external device may estimate a parameter inside a patient's bladder based on sensing distal to the patient.

As used herein, “sense” may include detect and/or measure. As used herein, “proximal” is used as defined in Section 3.1.4 of ASTM F623-19, Standard Performance Specification for Foley Catheter. That is, the proximal end of a catheter is the end closest to the patient when the catheter is being used by the patient. The distal end is therefore the end furthest from the patient.

As mentioned above, dissolved oxygen in urine of a patient can be relatively difficult to measure. One way to measure dissolved oxygen is by fluorescence or luminescence lifetime sensor(s). The decay of glow is indicative of the level of oxygen in urine of a patient. To more accurately measure the level of oxygen in the urine, it may be desirable to take the measurement prior to any significant modification in the oxygen content in the urine, e.g., as close to the kidneys as possible. However, it may not be feasible to place a dissolved oxygen sensor at the proximal end of the catheter as doing so may increase cost, size, and flexibility of the catheter.

Some Foley catheters include an elongated body made from a silicone rubber or another material that is relatively permeable to oxygen. Thus, as a fluid flows through a drainage lumen of the Foley catheter from a proximal opening (e.g., in a bladder of the patient) to the drainage lumen to a distal opening to the drainage lumen, some oxygen may permeate from the surrounding environment through the walls of the elongated body into urine in the drainage lumen or dissipate through the walls of the elongated body and into a surrounding environment. The flow rate of the fluid through the drainage lumen may impact the exchange of oxygen or other substance of interest between the drainage lumen and the exterior of the catheter. Slower fluid transit times (e.g., rate of flow) through the drainage lumen may result in erroneous or skewed measurements as the oxygen may permeate through the walls of the Foley catheter into the urine or dissipate from the urine through the walls of the Foley catheter as the urine travels from the bladder through the lumen. For example, oxygen may permeate into the urine through the walls of the Foley catheter as the urine travels through the lumen from the bladder to the oxygen sensor. As another example, the oxygen may dissipate into, out of, or permeate from other tissues in or near the urinary tract and the atmosphere outside of the urinary tract. In some examples, the devices and techniques described herein enable a device to determine, through the use of an observer and from an oxygen sensor signal positioned relatively far away from the fluid source, such as a bladder, an amount of dissolved oxygen in a fluid at a location within a patient, such as the bladder.

In some examples, rather than integrating all of the desired sensors in the proximal portion of an elongated body of a catheter (e.g., the portion that is to be inserted into the bladder of the patient or otherwise introduced in a patient), one or more sensors may be positioned anywhere along the elongated body (e.g., on the proximal portion or a distal portion) or distal to a distal end of the elongated body. For example, an oxygen sensor and/or a flow sensor may be located on a distal portion of the elongated body of the catheter or distal to a distal end of the elongated body of the catheter. The distal portion of the elongated body may include, for example, the portion intended to remain outside of the patient when the proximal portion is introduced in the patient. By locating sensors at the distal portion of the catheter or distal to a distal end of the elongated body, the sensors may be larger, may rely upon relatively more electrical and/or optical connections and the catheter itself may be smaller and more flexible than it would have been had all the sensors been positioned at the proximal portion of the catheter.

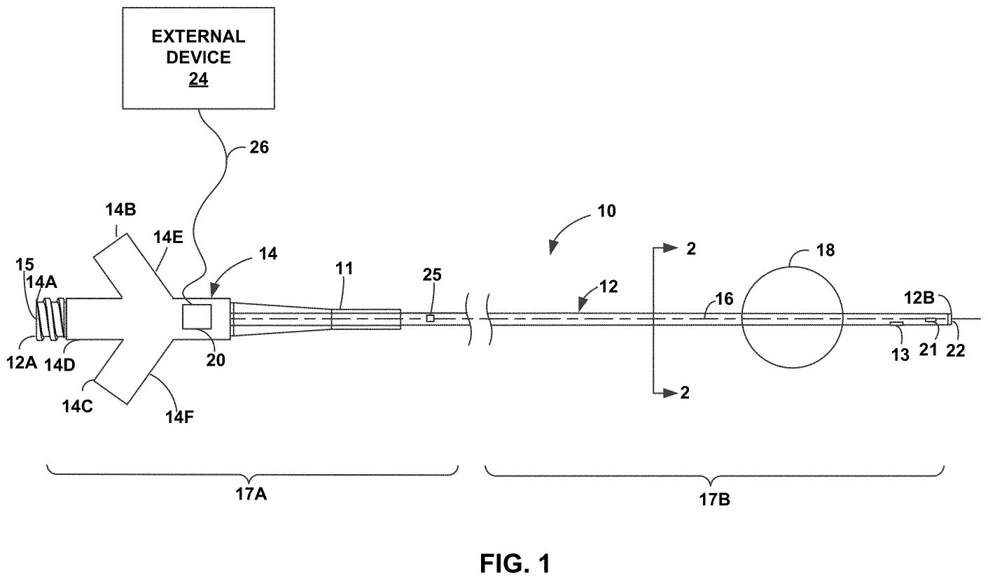

FIG. 1 is a conceptual side elevation view of an example catheter 10, which includes elongated body 12, hub 14, and anchoring member 18. In some examples, catheter 10 is a Foley catheter. While a Foley catheter and its intended use are primarily referred to herein to describe catheter 10, in other examples, catheter 10 can be used for other purposes, such as to drain wounds or for intravascular monitoring or medical procedures.

Catheter 10 includes a distal portion 17A and a proximal portion 17B. Distal portion 17A includes a distal end 12A of elongated body 12 and is intended to be external to a patient's body when in use, while proximal portion 17B includes a proximal end 12B of elongated body 12 and is intended to be internal to a patient's body when in use. For example, when proximal portion 17B is positioned within a patient, e.g., such that proximal end 12B of elongated body 12 is within the patient's bladder, distal portion 17A may remain outside of the body of the patient.

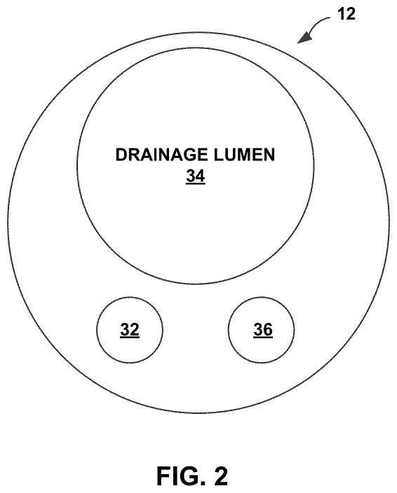

Elongated body 12 is a structure (e.g., a tubular structure) that extends from distal end 12A to proximal end 12B and defines one or more inner lumens. In the example shown in FIGS. 1-2, elongated body 12 defines lumen 32, drainage lumen 34 and anchoring lumen 36 (shown in FIG. 2). In other examples, elongated body 12 may define only one lumen, only two lumens (e.g., drainage lumen 34 and anchoring lumen 36) or more than three lumens. In some examples, drainage lumen 34 is configured to drain a fluid from a target site, such as a bladder. In other examples drainage lumen 34 may be used for any other suitable purpose, such as to deliver a substance or another medical device to a target site within a patient. Drainage lumen 34 may extend from a proximal fluid opening 13 to a distal fluid opening 14A. Both fluid opening 13 and fluid opening 14A may be fluidically coupled to drainage lumen 34, such that a fluid may flow from one of fluid opening 13 or fluid opening 14A to the other of fluid opening 13 or fluid opening 14A through drainage lumen 34. Fluid opening 13 and fluid opening 14A may also be referred to as drainage openings.

In some examples, lumen 32 (shown in FIG. 2) may be an injection lumen configured to deliver a fluid to a target site, such as a bladder. In other examples, lumen 32 may house sensor 21, or may be used for any other suitable purpose. Lumen 32 may extend from distal fluid opening 14C to proximal fluid opening 22. Both fluid opening 14C and fluid opening 22 may be fluidically coupled to lumen 32, such that a fluid may flow from one of fluid opening 14C or fluid opening 22 to the other of fluid opening 14C or fluid opening 22 through lumen 32. In the examples in which lumen 32 is an injection lumen, fluid opening 14C and fluid opening 22 may be injection openings. While fluid opening 22 is shown at the proximal end 12B of elongated body 12, fluid opening 22 may be positioned elsewhere on proximal portion 17B proximal to anchoring member 18.

Anchoring lumen 36 (shown in FIGS. 2) is configured to transport a fluid, such as sterile water or saline, or a gas, such as air, from anchoring opening 14B to anchoring member 18. For example, an inflation device (not shown) may pump fluid or gas into anchoring lumen 36 through anchoring opening 14B into anchoring member 18 such that anchoring member 18 is inflated to a size suitable to anchor catheter 10 within the patient's bladder. In examples in which anchoring member 18 does not include an expandable balloon, rather than defining anchoring lumen 36, elongated body 12 may define an inner lumen configured to receive a deployment mechanism (e.g., a pull wire or a push wire) for deploying an expandable structure anchoring member 18 and hub 14 may comprise fluid opening 14A, fluid opening 14C and anchoring opening 14B via which a clinician may access the deployment mechanism.

In some examples, elongated body 12 has a suitable length for accessing the bladder of a patient through the urethra. The length may be measured along central longitudinal axis 16 of elongated body 12. In some examples, elongated body 12 may have an outer diameter of about 12 French to about 14 French, but other dimensions may be used in other examples. Distal portion 17A and proximal portion 17B of elongated body 12 may each have any suitable length.

In the example shown in FIG. 1, distal end 12A of elongated body 12 is received within hub 14 and is mechanically connected to hub 14 via an adhesive, welding, or another suitable technique or combination of techniques. Hub 14 is positioned at a distal end of elongated body 12 and defines an opening through which the one or more inner lumens (e.g., lumen 32, drainage lumen 34 and anchoring lumen 36, shown in FIG. 2) of elongated body 12 may be accessed and, in some examples, closed. While hub 14 is shown in FIG. 1 as having three arms, 14D, 14E and 14F, hub 14 may have any suitable number of arms, which may depend on the number of inner lumens defined by elongated body 12. For example, each arm may be fluidically coupled to a respective inner lumen of elongated body 12. In the example of FIG. 1, hub 14 comprises a fluid opening 14A, which is fluidically coupled to drainage lumen 34, an anchoring opening 14B, which is fluidically coupled to anchoring lumen 36, and a fluid opening 14C which is fluidically coupled to lumen 32 (shown in FIG. 2) of elongated body 12. In examples in which anchoring member 18 does not include an expandable balloon but includes an expandable structure configured to be expanded via a deployment mechanism (e.g., a pull wire or a push wire), the deployment mechanism may extend through lumen 32 and anchoring opening 14B.

In examples in which catheter 10 is a Foley catheter, a fluid collection container (e.g., a urine bag) may be attached to fluid opening 14A for collecting urine draining from the patient's bladder. Anchoring opening 14B may be operable to connect to an inflation device to inflate or otherwise expand anchoring member 18 positioned on proximal portion 17B of catheter 10. Hub 14 may include connectors, such as connector 15, for connecting to other devices, such as the fluid collection container or the inflation source. Fluid opening 14C may be operable to connect to an injection device or pull device, such as a pump, for injecting fluid into the patient's bladder or for pulling fluid out of patient's bladder. In some examples, catheter 10 includes strain relief member 11, which may be a part of hub 14 or may be separate from hub 14.

Proximal portion 17B of catheter 10 comprises anchoring member 18, fluid opening 13, fluid opening 22 and, in some examples, sensor 21. In some examples, sensor 21 is contained within lumen 32. Anchoring member 18 may include any suitable structure configured to expand from a relatively low-profile state to an expanded state in which anchoring member 18 may engage with tissue of a patient (e.g., inside a bladder) to help secure and prevent movement of proximal portion 17B out of the body of the patient. For example, anchoring member 18 can include an anchor balloon or other expandable structure. Anchoring member 18 may be uninflated or undeployed when not in use. When inflated or deployed, anchoring member 18 may function to anchor catheter 10 to the patient, for example, within the patient's bladder. In this manner, the portion of catheter 10 on the proximal side of anchoring member 18 may not slip out of the patient's bladder. Fluid opening 13 may be positioned on the surface of elongated body 12 between anchoring member 18 and the proximal end 12B (as shown) or may be positioned at the proximal end 12B. Fluid opening 22 may be positioned at the proximal end 12B (as shown) of elongated body 12 or may be positioned on the surface of elongated body between anchoring member 18 and the proximal end 12B.

Sensor 20 is positioned on distal portion 17A, such as on hub 14. In some examples, sensor 20 is alternatively positioned distal to distal end 12A, such as on additional tubing or another structure connected to hub 14. Sensor 20 is configured to sense and generate a signal indicative of a parameter of interest in a fluid, such as urine. The parameter of interest can be a substance of interest in the fluid, such as dissolved oxygen, or a flow rate of the fluid. The fluid can be, for example, fluid in drainage lumen 34 or fluid received from drainage lumen 34, such as distal to distal end 12A.

Sensor 20 may be positioned on hub 14, as shown, or may be positioned elsewhere on distal portion 17A of the body of catheter 10, or may be positioned distal to distal end 12A, e.g., on tubing connected to a fluid collection container (e.g., a urine bag) or the like. Sensor 20, may be one or more sensors that are relatively larger, require relatively more electrical, optoelectrical, or optical connections, than sensors that could be located on the proximal portion 17B. While sensor 20 is primarily discussed herein as sensing dissolved oxygen (oxygen tension or uPO2) and/or fluid output, in some examples, sensor 20 may additionally or alternatively include sensor(s) configured to sense one or more of temperature, pressure, fluid concentration, amount of dissolved carbon dioxide in the fluid, turbidity, fluid pH, fluid color, fluid creatinine, and/or motion. While shown in FIG. 1 as a single sensor, in some examples, sensor 20 may be a plurality of sensors, such as an oxygen sensor and a flow sensor.

In some examples, sensor 20 is mechanically connected to elongated body 12 or another part of catheter 10 using any suitable technique, such as, but not limited to, an adhesive, welding, by being embedded in elongated body 12, via a crimping band or another suitable attachment mechanism or combination of attachment mechanisms. As discussed above, in some examples, sensor 20 is not mechanically connected to elongated body 12 or catheter 10, but is instead mechanically connected to a structure that is distal to distal end 12A of catheter 10, such as to tubing that extends between hub 14 and a fluid collection container or is inserted into a lumen of catheter 10, such as drainage lumen 34 or lumen 32.

In some examples, sensor 20 includes an oxygen sensor configured to communicate an oxygen sensor signal indicative of an amount of dissolved oxygen in a fluid, such as urine, to external device 24. In some examples, sensor 20 may also include a flow sensor configured to communicate a flow sensor signal indicative of a flow rate of the fluid to external device 24. External device 24 may be a computing device, such as a workstation, a desktop computer, a laptop computer, a smart phone, a tablet, a server or any other type of computing device that may be configured to receive, process and/or display sensor data. Sensor 20 may communicate sensor data to the external device via a connection 26. Connection 26 may be an electrical, optical, wireless or other connection.

Although only sensor 20 and sensor 21 are shown in FIG. 1, in other examples, catheter 10 can include any suitable number of sensors on proximal portion 17B and any suitable number of sensors on distal portion 17A, where the sensors on proximal portion 17B sense the same or different parameters and the sensors on distal portion 17A sense the same or different parameters. In addition, some or all of the sensors on proximal portion 17B can sense the same or different parameters as the sensors on distal portion 17A. For example, in the case where sensors on the distal portion may be temperature dependent, it may be desirable to sense temperature both on the proximal portion 17B and the distal portion 17A.

Elongated body 12 may be structurally configured to be relatively flexible, pushable, and relatively kink- and buckle-resistant, so that it may resist buckling when a pushing force is applied to a relatively distal portion of elongated body 12 to advance elongated body 12 proximally through the urethra and into the bladder. Kinking and/or buckling of elongated body 12 may hinder a clinician's efforts to push the elongated body proximally.

In some examples, at least a portion of an outer surface of elongated body 12 includes one or more coatings, such as an anti-microbial coating, and/or a lubricating coating. The lubricating coating may be configured to reduce static friction and/kinetic friction between elongated body 12 and tissue of the patient as elongated body 12 is advanced through the urethra.

FIG. 2 is a diagram illustrating an example cross-section of elongated body 12 of catheter 10, where the cross-section is taken along line 2-2 in FIG. 1 in a direction orthogonal to central longitudinal axis 16. FIG. 2 depicts a cross section of elongated body 12, which defines lumen 32, drainage lumen 34, and anchoring lumen 36. While lumen 32, drainage lumen 34, and anchoring lumen 36 are shown as circular in cross-section, they may have any suitable cross-sectional shape in other examples.

Elongated body 12 can define any suitable number of lumens. For example, although one anchoring lumen 36 is shown in FIG. 2, in other examples, elongated body 12 can define a plurality of anchoring lumens 36, e.g., that are distributed around lumen 32 or drainage lumen 34. As another example, anchoring member 18 may be an expandable structure that is not an inflatable balloon. In such examples, anchoring lumen 36 may be replaced by a deployment mechanism which may permit a clinician to expand the expandable structure. For example, anchoring lumen 36 may be replaced by a mechanical device that may be pushed and pulled separately from the catheter 10 by a clinician to expand or retract the expandable structure. As another example of a different lumen configuration, in some examples, elongated body 12 may not include lumen 32 and can have only drainage lumen 34 and anchoring lumen 36.

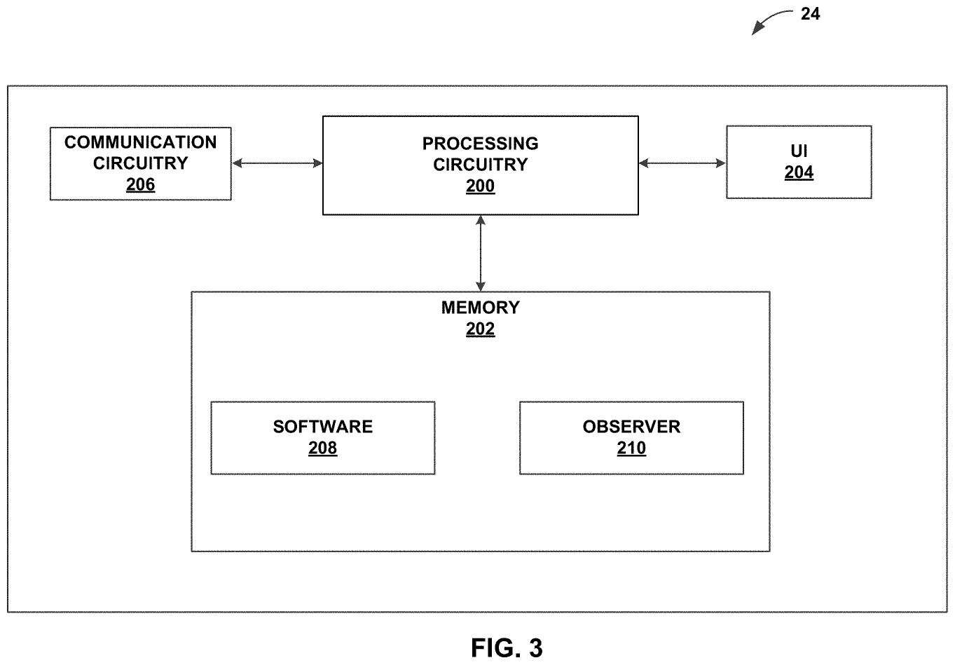

FIG. 3 is a functional block diagram illustrating an example of an external device 24 configured to communicate with sensor 20 (FIG. 1), receive information from sensor 20, such as an oxygen sensor signal indicative of an amount of dissolved oxygen in a fluid and/or a flow sensor signal indicative of a flow rate of the fluid. In some examples, external device 24 also is configured to communicate with or receive information from sensor 21 (FIG. 1). In some examples, sensor 21 may be a flow sensor configured to sense a flow rate of a fluid and communicate a flow sensor signal indicative of a flow rate of the fluid to external device 24. In the example of FIG. 3, external device 24 includes processing circuitry 200, memory 202, user interface (UI) 204, and communication circuitry 206. External device 24 may be a dedicated hardware device with dedicated software for reading sensor data. Alternatively, external device 24 may be an off-the-shelf computing device, e.g., a desktop computer, a laptop computer, a tablet, or a smartphone running a mobile application that enables external device 24 to read sensor data from sensor 20.

In some examples, a user of external device 24 may be clinician. In some examples, a user uses external device 24 to monitor a patient's kidney function. In some examples, the user may interact with external device 24 via UI 204, which may include a display to present a graphical user interface to the user and/or sound generating circuitry configured to generate audio output, and a keypad or another mechanism (such as a touch sensitive screen) configured to receive input from the user. External device 24 may communicate with sensor 20 or sensor 21 using wired, wireless or optical methods through communication circuitry 206. For example, processing circuitry 200 of external device 24 may process sensor data from sensor 20 or sensor 21.

Processing circuitry 200 may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 200 may include multiple components, such as any combination of one or more microprocessors, one or more DSPs, one or more ASICs, or one or more FPGAs, as well as other discrete or integrated logic circuitry, and/or analog circuitry.

Memory 202 may store program instructions, such as software 208, which may include one or more program modules including instructions, which are executable by processing circuitry 200. Memory 202 may store observer 210, which in some examples may be part of software 208. When executed by processing circuitry 200, such program instructions may cause processing circuitry 200, and external device 24 to provide the functionality ascribed to them herein. The program instructions may be embodied in software and/or firmware. Memory 202 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

This disclosure describes techniques and devices configured to aid in the monitoring of the one or both kidneys of a patient. In some examples, processing circuitry 200 of external device 24 monitors the amount of oxygen dissolved in the urine pO₂ in the bladder as it has been shown that this measurement reflects the oxygenation of the kidneys. To do this, the urine flow rate and the amount of oxygen dissolved in the urine may be sensed or measured. In some examples, the amount of oxygen dissolved in the urine is sensed at distal portion 17A of catheter 10 or distal to distal end 12A. Example techniques of this disclosure utilize a Foley catheter and sensors to make these measurements. In some examples, the sensors are part of the Foley catheter. In other examples, the sensors are not part of the Foley catheter.

To measure the pO₂ at the distal portion 17A of catheter 10 or distal to distal end 12A (away from the patient), processing circuitry 200 is configured to account for oxygen that diffuses from the environment into the urine in drainage lumen 34 or from the urine in drainage lumen 34 into the environment. As a result of such oxygen diffusion into or out of drainage lumen 34, the oxygen content of urine or another fluid in drainage lumen 34 may not accurately represent the oxygen content of urine in the bladder of the patient. Urine monitoring systems (e.g., processing circuitry 200 specifically) described herein are configured to estimate the oxygen content (e.g., pO₂) in the bladder of the patient through the use of an observer even though the oxygen content of the fluid is sensed at the distal portion 17A of catheter 10 or distal to distal end 12A.

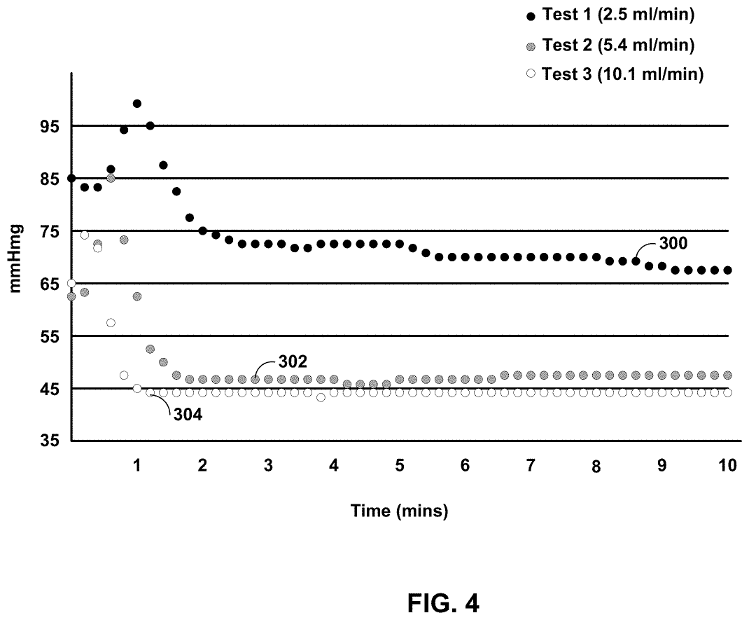

FIG. 4 is a graph illustrating pO₂ measurements of water that was initially set to ˜43 millimeters of mercury (mmHg) and allowed to flow through a silicone Foley catheter at different flow rates. As shown in FIG. 4, the pO₂ pick up (e.g., oxygen coming into the water through the catheter wall) from water with an initial pO₂ of ˜42 mmHg flowing through a silicone Foley Catheter at flow rates of 2.5 (line 300), 5.4 (line 302), and 10.1 (line 304) milliliters (mL)/minute (min) and a measured pO₂ pick up of 26.3, 6.4 and 3 mmHg, respectively, of the flow rates. Some urine flow rates range from 0-5 mL/min for catheterized patients. This indicates the pO₂ pick up could be significant for silicone catheters.

Patients can be catheterized during and after major surgery using an indwelling urinary (e.g., Foley) catheter (e.g., catheter 10) inserted into the bladder via the urethra. Oxygen may be sensed at the distal portion 17A of catheter 10 or distal to distal end 12A using an oxygen sensor (e.g., sensor 20) inserted in the flow stream between the catheter and the urine collecting bag or within a lumen of catheter 10, such as drainage lumen 34. As mentioned above, commercially available Foley catheters are oxygen permeable in varying degrees depending on the material from which the catheter was constructed. This results in diffusion of oxygen between the urine in catheter 10 and the ambient air as well as the urethra over the catheter wall. Furthermore, the catheter wall constitutes an oxygen buffer which takes up/releases oxygen from/to the urine. These mechanisms result in an alteration of the pO₂ from the true value in the bladder over the length of the catheter to the sample point at or near distal end 12A of catheter 10 where sensor 20 senses the pO₂. The degree of equilibration between the oxygen in the urine and the oxygen surrounding catheter 10 can be affected by: 1) the catheter material; 2) the inner and outer diameter of the catheter; 3) the wall thickness of the catheter; 4) the length of the catheter; 5) the portion of the catheter situated in the urethra and in the ambient air, respectively; 6) the flow rate of the urine—high flow rate results in a short transit time and thus, lower equilibration with the exterior oxygen concentration; 7) the change in flow rate; 8) the change in the oxygen partial pressure in the urine; and 9) temperature of the ambient air and temperature of the urine. Items 7 and 8 will affect the sensed pO₂ as catheter 10—as mentioned—acts like a buffer, thus resulting in an equilibration time to the new flow/oxygen condition in the urine.

Silicone Foley catheters may have a relatively high oxygen permeability resulting in a relatively high degree of oxygen equilibration with the oxygen at the outside wall of the Foley catheter. Latex Foley catheters have a lower, but still potentially significant, oxygen equilibration with the oxygen at the outside wall of the catheter. Polyvinyl chloride (PVC) Foley catheters have relatively low oxygen permeability, but have the draw back that they are stiffer than the silicone and latex catheters, which may result in a lower degree of patient comfort and convenience. It may be desirable to allow a clinician to use a commercially available Foley catheter type and length of their own choice in combination with an oxygen sensor inserted between the catheter and a urine collection bag or within a lumen of the Foley catheter. Thus, according to the techniques of this disclosure processing circuitry 200 may receive from sensor 20, an oxygen sensor signal indicative of an amount of dissolved oxygen in a fluid. Processing circuitry 200 may determine, based on the oxygen sensor signal, a measurement of the amount of dissolved oxygen in the fluid. Processing circuitry 200 may apply an observer to the measurement of the amount of dissolved oxygen in the fluid. Processing circuitry 200 may determine, based on the observer, an estimate of an amount of dissolved oxygen in the fluid at the location (e.g., bladder) within the patient. In this manner, a clinician may select a commercially available Foley catheter of their choice in combination with an oxygen sensor and receive an estimate of the amount of dissolved oxygen in the fluid at the location within the patient.

The measurement of the oxygen partial pressure in the urine can be confounded to a varying degree depending on the choice of catheter type, length and flow speed. pO₂ at the distal end of the catheter can be sensed, flow can be sensed, but it may be relatively difficult to directly sense the oxygen partial pressure at the proximal end of the catheter with a sensor at the distal end (e.g., sensor 20 (FIG. 1)) due to the oxygen permeability of catheter 10 (FIG. 1).

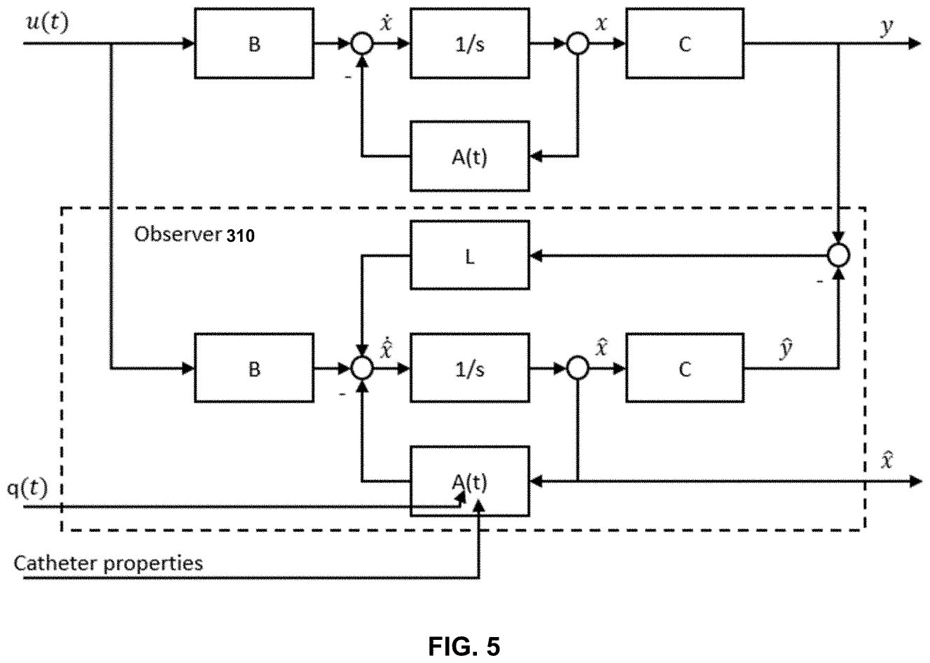

According to example techniques of this disclosure, processing circuitry of a device, such as external device 24 (FIG. 3), is configured to estimate the oxygen partial pressure at proximal end 12B of catheter 10 (FIG. 1) (e.g., in the bladder) based on observer 210 (FIGS. 3 and 5) (which may also be called an estimator) using control theory. For example, an observer may be used when the internal states of a system cannot be directly observed. Observer 210 uses mathematical relations to estimate the state of the catheter using other data such as: 1) the oxygen partial pressure in the urine at the distal end of the catheter; 2) oxygen contained in the catheter wall (which may be estimated based on material used to construct the catheter, the geometry of the catheter, and/or history of the catheter); and, in some examples, 3) fluid flow into the catheter (e.g., as indicated by a fluid flow rate in a lumen of catheter 10).

For example, in some examples, the input to observer 210 may include measured pO₂ at or near distal end 12A of catheter 10 and the measured urine flow rate. In some examples, the observer may be static and require a user, such as a clinician, to enter the type of catheter in order to determine the system matrices including: 1) Oxygen permeability of the catheter; 2) Length of the catheter; 3) Buffer capability of the catheter; and 4) Catheter volume (e.g., urine).

In some examples, the observer may be adaptive such that the observer adjusts itself to determine the system matrices for the used catheter. In some examples, the observer may be a combination of static and adaptive, e.g., the clinician may enter the catheter material on a device, such as external device 24, which may facilitate the adaptive portion of the observer to converge faster.

FIG. 5 is a conceptual diagram of an example observer. Observer 310 may be an example of observer 210 (FIG. 3). In the example of FIG. 5, u(t) is an input vector to observer 310 including: 1) the amount of dissolved oxygen in the urine inside the bladder; and 2) the fluid flow into the catheter; x is the state vector (oxygen partial pressure in the bladder and oxygen contained in the catheter wall); y is the measured oxygen partial pressure at the distal end of the catheter; and the system matrix A(t) represents a system matrix for the coupled system including the catheter, the fluid in the majority of the lumen of the catheter, and the fluid in proximity of the oxygen sensor element. B represents the input matrix and C represents the output matrices. B, C and D are in general time-invariant.

For the observer, A(t), B, C and D represent the same matrices for the system which may be determined by laboratory experiments, with input from a clinician, or with adaptive estimation of parameters from readings at different flow rates. q(t) represents the fluid flow into a lumen of the catheter, which may be sensed by a fluid flow sensor and fed into the observer system matrix to adjust A(t) according to the sensed fluid flow rate. Also, catheter properties corresponding to the chosen catheter may be input into the observer to adjust A(t). \hat{x} represents the estimated state vector of which the oxygen partial pressure inside the patient may be read out from the observer as the sought oxygen partial pressure. ŷ represents estimated oxygen partial pressure at the oxygen sensor location, which is subtracted from the measured oxygen partial pressure and fed into the observer via the observer gain matrix L.

In some examples, observer 310 may operate using the following equations:

\dot{x}(t)=A(t)x(t)+Bu(t)

y(t)=Cx(t)+Du(t)

\hat{\dot{x}}(t)=A(t)\hat{x}(t)+Bu(t)+L(y−ŷ)

ŷ(t)=C\hat{x}(t)+Du(t)

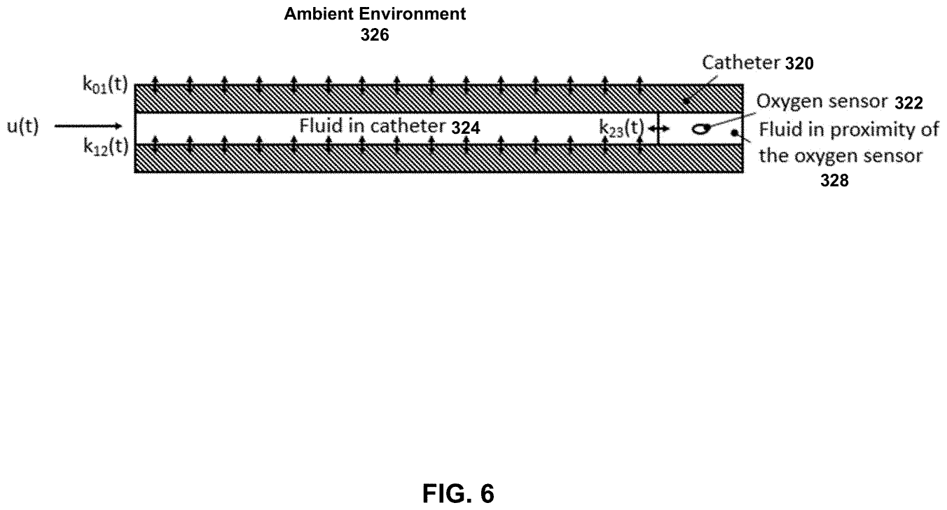

FIG. 6 is a conceptual diagram illustrating a portion of an example catheter. Catheter 320 may be an example of catheter 10 of FIG. 1. FIG. 6 illustrates catheter 320 including oxygen sensor 322 and fluid in catheter 324, which is shown as positioned in a lumen of catheter 320. In some examples, fluid in catheter 324 is urine. Fluid in catheter 324 may include fluid in proximity of oxygen sensor 328. Outside of catheter 320 is ambient environment 326. Ambient environment 326 may include tissue of a patient, such as urethra tissue, ambient air, or the like. k₀₁ is the oxygen coupling coefficient between catheter 320 and the ambient environment 326; k₁₂ is the oxygen coupling coefficient between catheter 320 and fluid in catheter 324, and k₂₃ is the coupling coefficient between the majority of fluid in catheter 324 and fluid in proximity of oxygen sensor 328. In some examples, the coupling coefficient k₂₃ may be highly dependent on the fluid flow rate, e.g., the transit time in the catheter from a fluid opening in the bladder of the patient to the location of oxygen sensor 322. For example, a relatively high fluid flow rate may result in low coupling whereas a relatively slow or no flow rate may result in high coupling. This results in the system matrix A(t) (FIG. 5) being time variant due to the varying coupling coefficient k₂₃. As the urine flow changes in a semi random manner covering a variety of flow speeds, thus probing the system (e.g., catheter 10) and observer 210 at a range of conditions and allowing for the observer to converge and provide an estimate for the true oxygen partial pressure in the bladder.

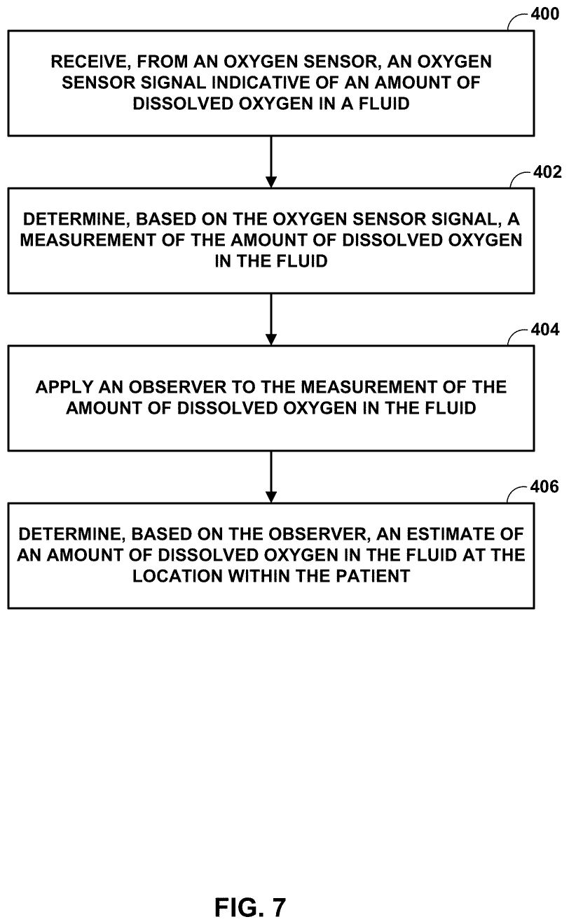

FIG. 7 is a flow diagram illustrating example oxygen estimation techniques according to this disclosure. Processing circuitry 200 may receive, from an oxygen sensor (e.g., sensor 20), an oxygen sensor signal indicative of an amount of dissolved oxygen in a fluid (400). The oxygen sensor is located in distal portion 17A of catheter 10 or distal to distal end 12A of catheter 10, and the fluid flows to the oxygen sensor from a location within a patient. For example, sensor 20 may sense pO₂ in urine either in drainage lumen 34 or distal to distal end 12A of catheter 10. Sensor 20 may send an oxygen sensor signal indicative of the pO₂ in the urine to communication circuitry 206 of external device 24 via connection 26. Communication circuitry 206 may send the oxygen sensor signal to processing circuitry 200. The urine may flow to sensor 20 from a bladder within the patient. For example, the location may be a bladder.

Processing circuitry 200 may determine, based on the oxygen sensor signal, a measurement of the amount of dissolved oxygen in the fluid (402). For example, processing circuitry 200 may calculate a measurement of the pO₂ in the urine based on the oxygen sensor signal. Processing circuitry 200 may apply observer 210 to the measurement of the amount of dissolved oxygen in the fluid (404). For example, processing circuitry 200 may invoke or execute observer 210 based on the measurement of the pO₂ in the urine as determined by processing circuitry 200. In other words, processing circuitry 200 may use the measured pO₂ as an input to observer 210. Processing circuitry 200 may determine, based on observer 210, an estimate of an amount of dissolved oxygen in the fluid at the location within the patient (406). For example, processing circuitry 200 may determine an estimated state variable of observer 210, from which the estimate of the pO₂ in the urine at the bladder within the patient may be derived. In some examples, processing circuitry 200 may also receive from a fluid sensor, a sensor signal indicative of a flow rate of an amount of flow of the fluid into the catheter and use the flow rate or amount of flow of the fluid as an input into observer 210.

In some examples, the observer 210 is a mathematical model of catheter 10 that indicates the estimate of the amount of dissolved oxygen in the fluid at the location within the patient based on an amount of dissolved oxygen in the fluid within catheter 10 or distal to distal end 12A of catheter 10. In some examples, processing circuitry 200 determines, e.g., based on input from a user or another device storing such information, at least one of an indication of oxygen permeability of catheter 10, a length of the catheter 10, a buffer capability of catheter 10, or a catheter volume (e.g., a volume of drainage lumen 34). For example, a user, such as a clinician, may input into UI 204, at least one of an indication of oxygen permeability of the catheter, a length of the catheter, a buffer capability of the catheter, or a catheter volume.

Processing circuitry 200 may use the clinician input with observer 210. In some examples, processing circuitry 200 enters the at least one of the indication of the oxygen permeability of catheter 10, the length of catheter 10, the buffer capability of catheter 10, or the catheter volume into the observer 210. The indication of oxygen permeability of catheter 10 may include any of, or any combination of, an actual oxygen permeability value, a material from which catheter 10 is constructed, a thickness of a wall of elongated body 12 of catheter 10 (e.g., between drainage lumen 34 and the external environment), a make and model of catheter 10, or the like. In some examples, memory 202 stores oxygen permeability values for different materials, thicknesses, makes and models, or the like and processing circuitry 200 determines an oxygen permeability value of catheter 10 based on the stored oxygen permeability values.

In some examples, processing circuitry 200 updates, based on at least one of sensed dissolved oxygen, flow rate of the fluid, or an amount of flow of the fluid, observer 210. In some examples, processing circuitry 200 receives, from a flow sensor (e.g., sensor 20 or sensor 21), a flow sensor signal indicative of a flow rate of the fluid through drainage lumen 34. In some examples, processing circuitry 200 determines, based on the flow sensor signal, a measurement of the flow rate the fluid through drainage lumen 34, wherein the estimate is further based on the measurement of the flow rate of the fluid. For example, processing circuity 200 may enter the determined flow rate of the urine into observer 210 and the output of observer 210 may be further based on the determined flow rate.

In some examples, processing circuitry 200 determines, based on the measurement of the flow rate of the fluid through drainage lumen 34, a measure of transit time of the fluid from the location within the patient (e.g., a bladder) through drainage lumen 34 of catheter 10 to the sensor 20. In some examples, processing circuitry 200 uses the transit time an input into observer 210. In some examples, as part of determining the estimate, processing circuitry 200 determines a material of elongated body 12 of catheter 10, wherein the estimate is further based on the material. For example, a user may enter the material through UI 204 or processing circuitry 200 may read the material from memory 202. For example, memory 202 may have stored a table including makes and models of catheters and associated materials. A user may enter the make and model through UI 204 and processing circuitry 200 may access the table to read the material associated with the entered make and model. In some examples, as part of applying observer 210, processing circuitry 200 may determine an amount of oxygen contained within a wall of the catheter.

Any of the techniques or examples described herein may be used alone or in combination with one or more other techniques or examples.

The techniques described in this disclosure, including those attributed to sensor 20, sensor 21, processing circuitry 200, communication circuitry 206, observer 210, and UI 204 or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples have been described. These and other examples are within the scope of the following claims.

What is claimed is:

1. A method comprising:
receiving, from an oxygen sensor, an oxygen sensor signal indicative of an amount of dissolved oxygen in a fluid, wherein the oxygen sensor is located in a distal portion of a catheter or distal to a distal end of the catheter, and wherein the fluid flows to the oxygen sensor from a location within a patient;
determining, by processing circuitry and based on the oxygen sensor signal, a measurement of the amount of dissolved oxygen in the fluid;
applying, by processing circuitry, an observer to the measurement of the amount of dissolved oxygen in the fluid; and
determining, by the processing circuitry and based on the observer, an estimate of an amount of dissolved oxygen in the fluid at the location within the patient.

2. The method of claim 1, wherein the observer comprises a mathematical model of the catheter that indicates the estimate of the amount of dissolved oxygen in the fluid at the location within the patient.

3. The method of claim 1, wherein the location is a bladder.

4. The method of claim 1, further comprising:
determining, based on input from a user, at least one of an indication of oxygen permeability of the catheter, a length of the catheter, a buffer capability of the catheter, or a catheter volume; and
entering the at least one of the indication of the oxygen permeability of the catheter, the length of the catheter, the buffer capability of the catheter, or the catheter volume into the observer.

5. The method of claim 1, further comprising:
updating, by the processing circuitry and based on at least one of sensed dissolved oxygen, flow rate of the fluid, or an amount of flow of the fluid, the observer.

6. The method of claim 1, further comprising:
receiving, from a flow sensor, a flow sensor signal indicative of a flow rate or amount of flow of the fluid; and
determining, by processing circuitry and based on the flow sensor signal, a measurement of the flow rate the fluid,
wherein the estimate is further based on the measurement of the flow rate or amount of flow of the fluid.

7. The method of claim 6, further comprising determining, by processing circuitry and based on the measurement of the flow rate of the fluid, a measure of transit time of the fluid from the location within the patient through a catheter to the oxygen sensor.

8. The method of claim 1, wherein determining the estimate comprises determining a material of the catheter, wherein the estimate is further based on the material of the catheter.

9. The method of claim 1, wherein applying the observer comprises:
determining an amount of oxygen contained within a wall of the catheter.

10. A device comprising:
memory configured to store an observer; and
processing circuitry communicatively coupled to the memory, the processing circuitry being configured to:
receive, from an oxygen sensor, an oxygen sensor signal indicative of an amount of dissolved oxygen in a fluid, wherein the oxygen sensor is located in a distal portion of a catheter or distal to a distal end of the catheter, and wherein the fluid flows to the oxygen sensor from a location within a patient;
determine, based on the oxygen sensor signal, a measurement of the amount of dissolved oxygen in the fluid;
apply the observer to the measurement of the amount of dissolved oxygen in the fluid; and
determine, based on the observer, an estimate of an amount of dissolved oxygen in the fluid at the location within the patient.

11. The device of claim 10, wherein the observer comprises a mathematical model of the catheter that indicates the estimate of the amount of dissolved oxygen in the fluid at the location within the patient.

12. The device of claim 10, wherein the location is a bladder.

13. The device of claim 10, wherein the processing circuitry is further configured to:
determine, based on input from a user, at least one of an indication of oxygen permeability of the catheter, a length of the catheter, a buffer capability of the catheter, or a catheter volume; and
enter the at least one of the indication of the oxygen permeability of the catheter, the length of the catheter, the buffer capability of the catheter, or the catheter volume into the observer.

14. The device of claim 10, wherein the processing circuitry is further configured to:
update the observer based on at least one of sensed dissolved oxygen, flow rate of the fluid, or an amount of flow of the fluid.

15. The device of claim 10, wherein the processing circuitry is further configured to:
receive, from a flow sensor, a flow sensor signal indicative of a flow rate or amount of flow of the fluid; and
determine, based on the flow sensor signal, a measurement of the flow rate or amount of flow of the fluid,
wherein the estimate is further based on the measurement of the flow rate or amount of flow of the fluid.

16. The device of claim 15, wherein the processing circuitry is further configured to:
determine, based on the measurement of the flow rate of the fluid, a measure of transit time of the fluid from the location within the patient through a catheter to the oxygen sensor.

17. The device of claim 10, wherein as part of determining the estimate, the processing circuitry is configured to: determine a material of the catheter, wherein the estimate is further based on the material of the catheter.

18. The device of claim 10, wherein as part of applying the observer, the processing circuitry is configured to:
determine an amount of oxygen contained within a wall of the catheter.

19. The device of claim 10, further comprising the oxygen sensor.

20. A non-transitory computer-readable storage medium having instructions stored thereon, which, when executed, cause processing circuitry to:
receive, from an oxygen sensor, an oxygen sensor signal indicative of an amount of dissolved oxygen in a fluid, wherein the oxygen sensor is located in a distal portion of a catheter or distal to a distal end of the catheter, and wherein the fluid flows to the oxygen sensor from a location within a patient;
determine, based on the oxygen sensor signal, a measurement of the amount of dissolved oxygen in the fluid;
apply an observer to the measurement of the amount of dissolved oxygen in the fluid; and
determine, based on the observer, an estimate of an amount of dissolved oxygen in the fluid at the location within the patient.
